(19) United States
(12) Patent Application Publication
(10) Pub. No.: US 2010/0063080 A1
(43) Pub. Date: Mar. 11, 2010

(54) CXCR2 inhibitors

(76) Inventors: Neil John Press, Horsham (GB); Peter Hunt, Horsham (GB); David Porter, Horsham (GB); Diana Janus, Horsham (GB)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/514,740

(22) PCT Filed: Nov. 21, 2007
(86) PCT No.: PCT/EP2007/010097

§ 371 Date: May 13, 2009

(30) Foreign Application Priority Data
Nov. 23, 2006 (EP) 06124683.1

(51) Int. Cl. A61K 31/505; C07D 239/46; A61P 29/00; A61P 11/00

(52) U.S. Cl. 514/274; 544/311

(57) ABSTRACT

The present invention relates to compounds of formula (I) and the use of these compounds as pharmaceuticals, e.g. in preventing or treating a CXCR2 receptor mediated condition or disease.

The present invention relates to 5-pyrimidinocarbonitriles, e.g. compounds of formula (I), and uses thereof.

In one aspect the present invention provides the use of a compound of formula

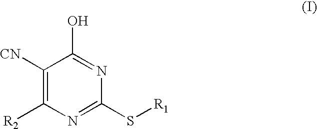

wherein
R₁ is (C₆₋₁₈)aryl or (C₆₋₁₈)aryl(C₁₋₄)alkyl, unsubstituted or one- or morefold substituted by (C₁₋₄)alkyl, C₁₋₄alkoxy, hydroxy, halogen,

### R₂ is

(C₁₋₈)alkyl, halo(C₁₋₈)alkyl, amino, substituted amino,
(C₆₋₁₈)aryl, unsubstituted or one- or morefold substituted by (C₁₋₄)alkyl, C₁₋₄)alkoxy, hydroxy or halogen,
(C₆₋₁₈)aryl(C₁₋₈)alkyl, unsubstituted or one- or morefold substituted,
heterocyclyl or heterocyclyl(C₁₋₈)alkyl, wherein heterocyclyl has 5 to 6 ring members and 1 to 4 heteroatoms selected from N, O, S, unsubstituted or one- or morefold substituted,
(C₃₋₈)cycloalkyl or (C₃₋₈)cycloalkyl(C₁₋₈)alkyl, unsubstituted or one- or morefold substituted,
   or a pharmaceutically acceptable salt or solvate thereof in the preparation of a medicament for the treatment of a CXCR2-related disorder or disease.

In another aspect the present invention provides the use of a compound of formula (I) as mentioned above wherein

R₁ is phenyl or phenyl(C₁₋₂)alkyl, unsubstituted or one- or morefold substituted by (C₁₋₄)alkyl, C₁₋₄)alkoxy, hydroxy, halogen,

### R₂ is

(C₁₋₄)alkyl, halo(C₁₋₄)alkyl, amino and substituted amino,
unsubstituted phenyl or phenyl substituted by (C₁₋₄)alkyl, C₁₋₄alkoxy, hydroxy, halogen, heterocyclyl,
phenyl(C₁₋₄)alkyl or phenyl(C₁₋₄alkyl, unsubstituted or one- or twofold substituted,
heterocyclyl or heterocyclyl(C₁₋₄)alkyl, wherein heterocyclyl has 5 to 6 ring members and 1 to 4 heteroatoms selected from N, O, S, unsubstituted or one- or twofold substituted,
(C₃₋₆)cycloalkyl or (C₃₋₆)cycloalkyl(C₁₋₄)alkyl, unsubstituted or one- or twofold substituted.

In another aspect the present invention provides the use of a compound of formula (I) as mentioned above, wherein

R₁ is unsubstituted benzyl or benzyl twofold substituted by fluoro,

### R₂ is

methyl, sec.butyl, tert.butyl, trifluoromethyl, amino substituted by 2-methylfuran-5-yl-1-propyl,
unsubstituted phenyl or phenyl one or twofold substituted by methyl, methoxy, iso-propoxy, hydroxy, chloro or thienyl,
1-phenyl-ethyl,
furanyl, 5-methyl-furan-2-yl-2-propyl, thienyl or pyridinyl,
unsubstituted cyclopropylmethyl,
unsubstituted cyclopropyl, cyclobutyl, cyclopentyl, cyclohexyl
cyclopropyl onefold substituted by ethoxycarbonyl, hydroxymethyl, hydroxycarbonyl, or a group of formula NR₃R₄—C═O—, wherein
   R₃ and R₄ independently are hydrogen, methyl, ethyl, 2-methyl-allyl, cyclopropylmethyl, methoxymethyl, ethoxycarbonylmethyl, hydroxymethyl, hydroxycarbonylmethyl, phenetyl, benzyl, benzyl substituted by methoxy, pyridin-3-yl-methyl, furan-2-yl-methyl, or
   R₃ and R₄ together form a 5 membered alicyclic ring system substituted by ethyl, a 6 membered alicyclic ring substituted by hydroxy, a 6 membered alicyclic ring having O as a further heteroatom, a 6 membered alicyclic ring having N as a further heteroatom which further N is substituted by tert.-butyloxycarbonyl.

If not otherwise defined herein
alkyl includes linear or branched (C₁₋₈)alkyl, such as (C₁₋₆)alkyl or (C₁₋₄)alkyl, e.g. (C₁₋₂)alkyl, including unsubstituted or substituted alkyl, e.g. alkyl substituted by groups which are conventional in organic chemistry, e.g. halogen, OH, NH₂ or halo(C₁₋₆)alkyl,
cycloalkyl includes (C₃₋₈)cycloalkyl, such as (C₃₋₈)cycloalkyl, e.g. cyclopropyl, cyclobutyl, cyclopentyl, cyclohexyl,
halogen includes fluoro, chloro, bromo, iodo, e.g. fluoro, chloro, bromo, preferably fluoro, chloro,
alkoxy includes (C₁₋₈)alkoxy, such as (C₁₋₄)alkoxy, e.g. methoxy, iso-propoxy,

aryl includes (C₆₋₁₈)aryl, e.g. phenyl, and (C₆₋₁₈)aryl, e.g. phenyl, annelated with heterocyclyl having 5 or 6 ring members and 1 to 4 heteroatoms selected from N, O, S
arylalkyl includes (C₆₋₁₈)aryl(C₁₋₈)alkyl, such as phenyl(C₁₋₈)alkyl, e.g. benzyl, phenethyl,
heterocyclyl includes heterocyclyl having 5 or 6 ring members and 1 to 4 heteroatoms selected from N, O, S, preferably N, O, such as alicyclic and aromatic heterocyclyl, e.g. heterocyclyl having 6 ring members and 1 to 2 heteroatoms selected from N, O, S, e.g. pyridinyl, such as pyridin-3-yl, pyridin-4-yl, thienyl, such as thien-2-yl, furanyl, e.g. furan-2-yl, furan-3-yl,

In a compound of formula (I) R₁ preferably is unsubstituted benzyl or benzyl twofold substituted by fluoro;

In a compound of formula (I) R₂ preferably is—methyl, sec.butyl, tert.butyl, trifluoromethyl, amino substituted by 2-methylfuran-5-yl-1-propyl,
unsubstituted phenyl or phenyl one or twofold substituted by methyl, methoxy, iso-propoxy, hydroxy, chloro, thienyl,
1-phenyl-ethyl,
furanyl, e.g. furan-2-yl, furan-3-yl; 5-methyl-furan-2-yl-2-propyl, thienyl, pyridinyl, e.g. pyridin-3-yl, pyridin-4-yl
unsubstituted cyclopropylmethyl,
unsubstituted cyclopropyl, cyclobutyl, cyclopentyl, cyclohexyl
cyclopropyl onefold substituted by ethoxycarbonyl, hydroxymethyl, hydroxycarbonyl, or a group of formula NR₃R₄—C═O—, wherein
R₃ and R₄ independently are hydrogen, methyl, ethyl, 2-methyl-allyl, cyclopropylmethyl, methoxymethyl, ethoxycarbonylmethyl, hydroxymethyl, hydroxycarbonylmethyl, phenetyl, benzyl, benzyl substituted by methoxy, pyridin-3-yl-methyl, furan-2-yl-methyl, or
R₃ and R₄ together form a 5 membered alicyclic ring system substituted by ethyl, a 6 membered alicyclic ring substituted by hydroxy, a 6 membered alicyclic ring having O as a further heteroatom, a 6 membered alicyclic ring having N as a further heteroatom which further N is substituted by tert.-butyloxycarbonyl.

In a compound of formula (I) each single defined substitutent may be a preferred substituent, e.g. independently of each other substitutent defined.

In another aspect the present invention provides a compound of formula (I) selected from the group consisting of
2-(2,3-Difluoro-benzylsulfanyl)-4-hydroxy-6-(4-methoxy-phenyl)-pyrimidine-5-carbonitrile
4-(3,4-Dichlorophenyl)-2-(2,3-difluorobenzylsulfanyl-6-hydroxy-pyrimidine-5-carbonitrile
2-(2,3-Difluorobenzylsulfanyl)-4-hydroxy-6-(4-hydroxyphenyl)-pyrimidine-5-carbonitrile
4-cyclopropyl-2-(2,3-difluorobenzylsulfanyl-6-hydroxypyrimidine-5-carbonitrile
2-(2,3-Difluoro-benzylsulfanyl)-4-hydroxy-6-phenyl-pyrimidine-5-carbonitrile
4-(4-Chlorophenyl)-2-(2,3-difluoro-benzyl-sulfanyl)-6-hydroxy-6-phenyl-pyrimidine-5-carbonitrile
4-(4-Methylphenyl)-2-(2,3-difluoro-benzyl-sulfanyl)-6-hydroxy-6-phenyl-pyrimidine-5-carbonitrile
2-(2,3-Difluoro-benzylsulfanyl)-4-furan-2-yl-6-hydroxy-pyrimidine-5-carbonitrile
2-(2,3-Difluoro-benzylsulfanyl)-4-hydroxy-6-pyridin-3-yl-pyrimidine-5-carbonitrile
2-(2,3-Difluoro-benzylsulfanyl)-4-hydroxy-6-pyridin-4-yl-pyrimidine-5-carbonitrile
2-(2,3-Difluoro-benzylsulfanyl)-4-hydroxy-6-(4-thiophen-2-yl-phenyl)-pyrimidine-5-carbonitrile
2-(2,3-Difluoro-benzylsulfanyl)-4-hydroxy-6-m-tolyl-pyrimidine-5-carbonitrile
2-(2,3-Difluoro-benzylsulfanyl)-4-furan-3-yl-6-hydroxy-pyrimidine-5-carbonitrile
2-(2,3-Difluoro-benzylsulfanyl)-4-hydroxy-6-(4-isopropoxy-phenyl)-pyrimidine-5-carbonitrile
4-(4-tert-Butyl-phenyl)-2-(2,3-difluoro-benzylsulfanyl)-6-hydroxy-pyrimidine-5-carbonitrile
2-(2,3-Difluoro-benzylsulfanyl)-4-(3,4-dimethyl-phenyl)-6-hydroxy-pyrimidine-5-carbonitrile
4-Cyclopentyl-2-(2,3-difluoro-benzylsulfanyl)-6-hydroxy-pyrimidine-5-carbonitrile
4-Cyclopropyl-2-(2,3-difluoro-benzylsulfanyl)-6-hydroxy-pyrimidine-5-carbonitrile
2-(2,3-Difluoro-benzylsulfanyl)-4-hydroxy-6-(4-methoxy-3-methyl-phenyl)-pyrimidine-5-carbonitrile
4-Cyclobutyl-2-(2,3-difluoro-benzylsulfanyl)-6-hydroxy-pyrimidine-5-carbonitrile
4-Cyclohexyl-2-(2,3-difluoro-benzylsulfanyl)-6-hydroxy-pyrimidine-5-carbonitrile
2-[5-Cyano-2-(2,3-difluoro-benzylsulfanyl)-6-hydroxy-pyrimidin-4-yl]-cyclopropanecarboxylic acid ethyl ester
2-(2,3-Difluoro-benzylsulfanyl)-4-ethyl-6-hydroxy-pyrimidine-5-carbonitrile
4-sec-Butyl-2-(2,3-difluoro benzyl sulfanyl)-6-hydroxy-pyrimidine-5-carbonitrile
4-tert-Butyl-2-(2,3-difluoro-benzylsulfanyl)-6-hydroxy-pyrimidine-5-carbonitrile
2-(2,3-Difluoro-benzylsulfanyl)-4-hydroxy-6-(1-phenyl-ethyl)-pyrimidine-5-carbonitrile
2-(2,3-Difluoro-benzylsulfanyl)-4-hydroxy-6-trifluoromethyl-pyrimidine-5-carbonitrile
2-(2,3-Difluoro-benzylsulfanyl)-4-hydroxy-6-methyl-pyrimidine-5-carbonitrile
4-sec-Butyl-2-(2,3-difluoro-benzyl sulfanyl)-6-hydroxy pyrimidine-5-carbonitrile single enantiomer E1
4-sec-Butyl-2-(2,3-difluoro-benzyl sulfanyl)-6-hydroxy pyrimidine-5-carbonitrile single enantiomer E2
2-(2,3-Difluoro-benzylsulfanyl)-4-hydroxy-6-(3-hydroxy-phenyl)-pyrimidine-5-carbonitrile-4-sec-Butyl-2-(2,3-difluoro-benzyl sulfanyl)-6-hydroxy pyrimidine-5-carbonitrile
2-(2,3-Difluoro-benzylsulfanyl)-4-hydroxy-6-(1-phenyl-ethyl)-pyrimidine-5-carbonitrile
4-Cyclopropylmethyl-2-(2,3-difluoro-benzylsulfanyl)-6 hydroxy-pyrimidine-5-carbonitrile
Trans-2-[5-Cyano-2-(2,3-difluoro-benzylsulfanyl)-6 hydroxy-pyrimidin-4-yl]cyclopropane carboxylic acid
(1R,2R)-2-[5-Cyano-2-(2,3-difluoro-benzylsulfanyl)-6 hydroxy-pyrimidin-4-yl]cyclopropane carboxylic acid
(1S,2S)-2-[5-Cyano-2-(2,3-difluoro-benzylsulfanyl)-6 hydroxy-pyrimidin-4-yl]cyclopropane carboxylic acid
Trans-2-(2,3-Difluoro-benzylsulfanyl)-4-hydroxy-6-(2-hydroxymethyl-cyclopropyl)-pyrimidine-5-carbonitrile
Trans-2-[5-Cyano-2-(2,3-difluoro-benzyl sulfanyl)-6-hydroxy-pyrimidin-4-yl]cyclopropane carboxylic acid ethyl-(2-methyl-allyl)-amide
2-[5-Cyano-2-(2,3-difluoro-benzylsulfanyl)-6-hydroxy-pyrimidin-4-yl]-cyclopropane carboxylic acid ethyl-methyl-amide
2-[5-Cyano-2-(2,3-difluoro-benzylsulfanyl)-6-hydroxy-pyrimidin-4-yl]-cyclopropane carboxylic acid cyclopropyl methyl-amide
2-(2,3-Difluoro-benzylsulfanyl)-4-[2-(2-ethyl-pyrrolidine-1-carbonyl)-cyclopropyl]-6-hydroxypyrimidine-5-carbonitrile
2-[5-Cyano-2-(2,3-difluoro-benzylsulfanyl)-6-hydroxy-pyrimidin-4-yl]-cyclopropane carboxylic acid (1-methoxymethyl-propyl)-amide
2-[5-Cyano-2-(2,3-difluoro-benzylsulfanyl)-6-hydroxy-pyrimidin-4-yl]-cyclopropane carboxylic acid phenethyl-amide
2-[5-Cyano-2-(2,3-difluoro-benzylsulfanyl)-6-hydroxy-pyrimidin-4-yl]-cyclopropane carboxylic acid 2-methoxy-benzylamide
2-(2,3-Difluoro-benzylsulfanyl)-4-hydroxy-6-[2-(3-hydroxy-piperidine-1-carbonyl)cyclopropyl]-pyrimidine-5-carbonitrile
2-[5-Cyano-2-(2,3-difluoro-benzylsulfanyl)-6-hydroxy-pyrimidin-4-yl]-cyclopropane carboxylic acid (pyridin-3-ylmethyl)-amide
Trans-({2-[5-Cyano-2-(2,3-difluoro-benzylsulfanyl)-6-hydroxy-pyrimidin-4-yl]-cyclopropane carbonyl}-amino)-acetic acid ethyl ester
2-(2,3-Difluoro-benzylsulfanyl)-4-hydroxy-6-[2-(morpholine-4-carbonyl)-cyclopropyl]pyrimidine-5-carbonitrile
2-[5-Cyano-2-(2,3-difluoro-benzylsulfanyl)-6-hydroxy-pyrimidin-4-yl]-cyclopropane carboxylic acid (furan-2-ylmethyl)-amide
2-[5-Cyano-2-(2,3-difluoro-benzylsulfanyl)-6-hydroxy-pyrimidin-4-yl]-cyclopropane carboxylic acid amide
Trans-4-{2-[5-Cyano-2-(2,3-difluoro-benzylsulfanyl)-6-hydroxy-pyrimidin-4-yl]-cyclopropane carbonyl}-piperazine-1-carboxylic acid tert-butyl ester
Trans-({2-[5-Cyano-2-(2,3-difluoro-benzylsulfanyl)-6-hydroxy-pyrimidin-4-yl]-cyclopropane carbonyl}-amino)-acetic acid
2-(2,3-Difluoro-benzylsulfanyl)-4-hydroxy-6-[(R)-2-(5-methyl-furan-2-yl)-propyl]-pyrimidine-5-carbonitrile
(1S,2S)-2-(2,3-Difluoro-benzylsulfanyl)-4-hydroxy-6-(2-hydroxymethyl-cyclopropyl)-pyrimidine-5-carbonitrile
2-(2,3-Difluoro-benzylsulfanyl)-4-hydroxy-6-[(R)-1-(5-methyl-furan-2-yl)-propylamino]-pyrimidine-5-carbonitrile and
(1R,2R)-2-(2,3-Difluoro-benzylsulfanyl)-4-hydroxy-6-(2-hydroxymethyl-cyclopropyl)-pyrimidine-5-carbonitrile.

Compounds of formula (I) in free or pharmaceutically acceptable salt form are hereinafter referred to alternatively as compounds of the invention.

A compound of the present invention may exist in the form of isomers and mixtures thereof; e.g. optical isomers, diastereoisomers, cis/trans isomers. A compound of the present invention may e.g. contain asymmetric carbon atoms and may thus exist in the form of enantiomers or diastereoisomers and mixtures thereof, e.g. racemates. Substituents at any asymmetric carbon atom may be present in the (R)-, (S)- or (R,S)-configuration, preferably in the (R)- or (S)-configuration. E.g. cis/trans isomers may be present, in case that an aliphatic double bond is present in a compound of the present invention. Isomeric mixtures may be separated as appropriate, e.g. according, e.g. analogously, to a method as conventional, to obtain pure isomers. The present invention includes a compound of the present invention in any isomeric form and in any isomeric mixture.

The present invention also includes tautomers of a compound of the present invention, e.g. a compound of the present invention may be present in the following forms:

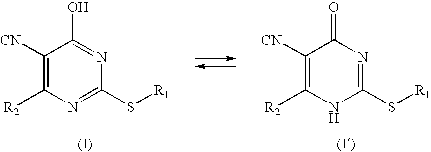

Any compound described herein, e.g. a compound of the present invention, may be prepared as appropriate, e.g. according, e.g. analogously, to a method as conventional, e.g. or as specified herein. Starting materials are known or may be prepared according, e.g. analogously, to a method as conventional or as described herein.

A compound of formula (I) thus obtained may be converted into another compound of formula (I), e.g. or a compound of formula I obtained in free form may be converted into a salt of a compound of formula (I) and vice versa.

Compounds of the invention, are useful as pharmaceuticals.

Accordingly the invention also provides a compound of formula (I) in free or pharmaceutically acceptable salt form for use as a pharmaceutical.

In another aspect the present invention provides the use of a compound of formula (I) wherein the substituents are as defined above as a pharmaceutical.

The compounds of the invention act as CXCR2 receptor antagonists, thereby inhibiting the infiltration and activation of inflammatory cells, in particular neutrophils, monocytes and CD8+ T cells and mediators involved in chronic obstructive pulmonary disease (COPD). The compounds of the invention therefore provide symptomatic relief and reduce disease progression.

The airways of subject with COPD exhibit an inflammatory response which is predominantly neutrophilic. When the airways are exposed to cigarette smoke macrophages, CD8+ T cells and epithelial cells are activated and release pro-inflammatory mediators, oxidants, cytokines and neutophilic chemotactic factors, IL-8, GROα, ENA-78 and leukotrienes. IL-8, GROα and ENA-78 are selective chemoattractants for neutrophils. In human neutrophils IL-8 binds two distinct receptors with similar affinity, CXCR1 and CXCR2. Closely related chemokines including GROα, β, γ, NAP-2 and ENA-78 bind only to CXCR2. Inhibiting neutrophil recruitment is therefore a recognised therapeutic strategy for treating several lung diseases. Blocking the binding of IL-8, GROα and ENA-78 to the chemokine receptor CXCR2 can provide beneficial effects in patients with COPD by suppressing the infiltration and activation of key inflammatory cells, thereby reducing subsequent tissue damage, mucus secretion, airflow obstruction and disease progression.

The IL-8 and GROα chemokine inhibitory properties of compounds of the invention can be demonstrated in the following ASSAYS:

### Receptor Binding Assay

[¹²⁵I] IL-8 (human recombinant) are obtained from Amersham Pharmacia Biotech, with specific activity 2000 Ci/mmol. All other chemicals are of analytical grade. Human recombinant CXCR2 receptor expressed in Chinese hamster ovary cells (CHO-K1) is purchased from Euroscreen. The Chinese hamster ovary membranes are prepared according to protocol supplied by Euroscreen. Membrane protein concentration is determined using a Bio-Rad protein assay. Assays are performed in a 96-well micro plate format according the method described in White, et al., J Biol. Chem., 1998, 273, 10095). Each reaction mixture contains 0.05 mg/ml CXCR2 membrane protein in 20 mM Bis-Tris-propane, pH 8.0, containing 1.2 mM MgSO₄, 0.1 mM EDTA, 25 mM NaCl and 0.03% CHAPS. In addition, compound of interest pre-dissolved in dimethylsulphoxide (DMSO) so as to reach a final concentration of between 10 μM and 0.0005 μM (final concentration of DMSO 2% (v/v)) is added. Binding is initiated by addition of 0.02 nM ¹²⁵I-IL-8. After 2 hours at room temperature the plate is harvested using a Brandell™ 96-well harvester onto glass fibre filter plate (GF/c) blocked with 1% polyethyleneimine+0.5% BSA and washed 3 times with 25 mM NaCl, 10 mM TrisHCl, 1 mM MgSO₄, 0.5 mM EDTA, 0.03% CHAPS, pH 7.4. The filter is dried at 50° overnight. Backseal is applied to the plate and 50 μl of liquid scintillation fluid added. The counts are measured on the Packard Topcount™ scintillation counter.

[³⁵S]-GTPγS Binding Assay for Human CXCR2 Receptor using SPA Technology

[³⁵S]-GTPγS (with specific activity 1082 Ci/mmol) and wheat germ agglutinin poly vinyl toluene scintillation proximity beads are purchased from Amersham Pharmacia Biotech. The Chinese hamster ovary cell (CHO-K1) membranes expressing human CXCR2 receptors are purchased from Biosignal Packard Inc. All other chemicals are of analytical grade. White non-binding surface 96 well Optiplate™ microplates are obtained from Packard. Recombinant human IL-8 is synthesised, cloned and expressed in Escherichia coli as described previously (Lindley I, et al., Proc. Natl. Acad. Sci., 1988, 85(23):9199). The assay is performed in duplicate in 96 well Optiplate™ microplate in a final volume of 250 μl per well. Compounds are diluted in DMSO (0.5% final concentration) and incubated in 20 mM HEPES buffer pH 7.4 containing 10 mM MgCl₂, 100 mM NaCl, 1 mM EDTA plus 100 nM IL-8, 50 μM GDP and 500 μM [³⁵S]GTPγS per well. SPA beads (1 mg/well final concentration) were pre-mixed with the membranes (10 μg/well final concentration) in assay buffer: 20 mM HEPES buffer pH 7.4 containing 10 mM MgCl₂, 100 mM NaCl, 1 mM EDTA. The bead membrane mixture is added to each well, plates are sealed and incubated at room temperature for 60 minutes. The plate is centrifuged and read on Packard TopCount™ scintillation counter, program [³⁵S dpm] for 1 min/well. Data are expressed as the % response to 100 nM IL-8 minus basal.

### Chemotaxis Assay

The in vitro inhibitory properties of these compounds are determined in the neutrophil chemotaxis assay. Assays are performed in a 96-well plate format according to previously published method (Frevert C W, et al., J Immunolog. Methods, 1998, 213, 41). 96-well chemotaxis chambers 5 μm are obtained from Neuro Probe, all cell buffers are obtained from Invitrogen Paisley, UK, dextran-T500 and Ficoll-Paque Plus™ density gradient centrifugation media are purchased from Pharmacia Biotech Buckinghamshire, UK. Calcein-AM dye is obtained from Molecular Probes. Neutrophils are isolated as previously described (Haslett, C., et al. Am J Path., 1985, 119:101). Citrated whole blood is mixed with 4% (w/v) dextran-T500 and allowed to stand on ice for 30 minutes to remove erythrocytes. Granulocytes (PMN) are separated from peripheral blood mononuclear cells by layering 15 ml of cell suspension onto 15 ml Ficoll-Paque PLUS density gradient and centrifuged at 250×g for 25 minutes. Following centrifugation any erythrocytes contamination of PMN pellet is removed by hypotonic shock lysis using 10 ml ice-cold endotoxin-free sterile water for 50 seconds and neutralised with 10 ml of cold 2× phosphate buffered saline. Isolated neutrophils (1×10⁷) are labelled with the fluorochrome calcein-AM (5 μg) in a total volume of 1 ml and incubated for 30 minutes at 37° C. The labelled cells are washed with RPMI without phenol red+0.1% bovine serum albumin, prior to use the cells are counted and adjusted to a final concentration of 5×10⁶ cells/ml. The labelled neutrophils are then mixed with test compounds (0.001-1000 nM) diluted in DMSO (0.1% final concentration) and incubated for 10 minutes at room temperature. The chemoattractants (29 μl) are placed in the bottom chamber of a 96-well chemotaxis chamber at a concentration between (0.1-5 nM). The polycarbonate filter (5 μm) is overlaid on the plate, and the cells (25 μl) are loaded on the top filter. The cells are allowed to migrate for 90 minutes at 37° C. in a humidified incubator with 5% CO₂. At the end of the incubation period, migrated cells are quantified using a multi-well fluorescent plate reader (Fluoroskan II™, Labsystems) at 485 nm excitation and 538 nm emission. Each compound is tested in quadruplet using 4 different donors. Positive control cells, i.e. cells that have not been treated with compound, are added to the bottom well. These represent the maximum chemotactic response of the cells. Negative control cells, i.e. those that have not been stimulated by a chemoattractant, are added to the bottom chamber. The difference between the positive control and negative control represents the chemotactic activity of the cells.

The compounds of the Examples herein below generally have IC₅₀ values below 2 μM in the [³⁵S]-GPTγS binding assay. For instance, the compounds of Examples 43 and 49 have IC₅₀ values of 230 nM and 820 nM, respectively.

Having regard to their inhibition of binding of CXCR2, compounds of the invention are useful in the treatment of conditions or diseases mediated by CXCR2, for example inflammatory or allergic conditions or diseases, particularly chronic obstructive pulmonary airways or lung disease (COPD, COAD or COLD), including chronic bronchitis or dyspnea associated therewith, emphysema, bronchiolitis obliterans syndrome and severe asthma.

Compounds of the present invention are further useful in the treatment of various diseases, such as cancer, e.g. ovarian cancer, prostate cancer, melanoma including metastatic melanoma, lung cancer, e.g. non small cell lung cancer, renal cell carcinoma; tumour angiogenesis, ischaemia/reperfusion injury, delayed graft function, osteoarthritis, myeloid metaplasia with myelofibrosis, Adenomyosis, contact hypersensitivity (skin) and in wound healing.

Treatment in accordance with the invention may be symptomatic or prophylactic.

Prophylactic efficacy in the treatment of chronic bronchitis or COPD will be evidenced by reduced frequency or severity, will provide symptomatic relief and reduce disease progression, improvement in lung function. It may further be evidenced by reduced requirement for other, symptomatic therapy, i.e. therapy for or intended to restrict or abort symptomatic attack when it occurs, for example anti-inflammatory (e.g. corticosteroid) or bronchodilatory.

Other inflammatory or obstructive airways diseases and conditions to which the invention is applicable include acute lung injury (ALI), acute/adult respiratory distress syndrome (ARDS), idiopathic pulmonary fibrosis, fibroid lung, airway hyperresponsiveness, dyspnea, pulmonary fibrosis, allergic airway inflammation, small airway disease, lung carcinoma, acute chest syndrome in patients with sickle cell disease and pulmonary hypertension, as well as exacerbation of airways hyperreactivity consequent to other drug therapy, in particular other inhaled drug therapy. The invention is also applicable to the treatment of bronchitis of whatever type or genesis including, e.g., acute, arachidic, catarrhal, croupus, chronic or phthinoid bronchitis. Further inflammatory or obstructive airways diseases to which the invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Compounds of the invention are also useful for treating respiratory viral infections, which exacerbate underlying chronic conditions such as asthma, chronic bronchitis, COPD, otitis media, and sinusitis. The respiratory viral infection treated may be associated with secondary bacterial infection, such as otitis media, sinusitis or pneumonia.

Compounds of the invention are also useful in the treatment of inflammatory conditions of the skin, for example psoriasis, atopic dermatitis, lupus erythematosus, and other inflammatory or allergic conditions of the skin.

Compounds of the invention may also be used for the treatment of other diseases or conditions, in particular diseases or conditions having an inflammatory component, for example, diseases affecting the nose including allergic rhinitis, e.g. atrophic, chronic, or seasonal rhinitis, inflammatory conditions of the gastrointestinal tract, for example inflammatory bowel disease such as ulcerative colitis and Crohn's disease, diseases of the bone and joints including rheumatoid arthritis, psoriatic arthritis, and other diseases such as atherosclerosis, multiple sclerosis, and acute and chronic allograft rejection, e.g. following transplantation of heart, kidney, liver, lung or bone marrow.

Compounds of the invention are also useful in the treatment of endotoxic shock, glomerulonephritis, cerebral and cardiac ischemia, Alzheimer's disease, cystic fibrosis, virus infections and the exacerbations associated with them, acquired immune deficiency syndrome (AIDS), multiple sclerosis (MS), Helicobacter pylori associated gastritis, and cancers, particularly the growth of ovarian cancer.

Compounds of the invention are also useful for treating symptoms caused by viral infection in a human which is caused by the human rhinovirus, other enterovirus, coronavirus, herpes viruses, influenza virus, parainfluenza virus, respiratory syncytial virus or an adenovirus.

Compounds of the invention are also useful for treating diseases such as pancreatitis, Behcet's disease and hepatobiliary diseases associated with reactive bile ductule, such as chronic viral hepatitis, liver cirrhosis, sepsis, extrahepatic biliary obstruction, fulminant hepatitis, primary biliary cirrhosis and primary sclerosing cholangitis.

The effectiveness of a compound of the invention in inhibiting inflammatory conditions, for example in inflammatory airways diseases, may be demonstrated in an animal model, e.g. mouse, rat or rabbit model, of airway inflammation or other inflammatory conditions, for example as described by Wada et al, J. Exp. Med. (1994) 180:1135-40; Sekido et al, Nature (1993) 365:654-57; Modelska et al., Am. J. Respir. Crit. Care. Med. (1999) 160:1450-56; and Laffon et al (1999) Am. J. Respir. Crit. Care Med. 160:1443-49.

The compounds of the invention are also useful as co-therapeutic compounds for use in combination with other drug substances such as anti-inflammatory, bronchodilatory, antihistamine or anti-tussive drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate, or steroids described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/35668, WO 03/48181, WO 03/62259, WO 03/64445, WO 03/72592, WO 04/39827 and WO 04/66920; non-steroidal glucocorticoid receptor agonists, such as those described in DE 10261874, WO 00/00531, WO 02/10143, WO 03/82280, WO 03/82787, WO 03/86294, WO 03/104195, WO 03/101932, WO 04/05229, WO 04/18429, WO 04/19935 and WO 04/26248; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID(TM) CC-10004 (Celgene), VM554/UM565 (Vernalis), T-440 (Tanabe), KW-4490 (Kyowa Hakko Kogyo), and those disclosed in WO 92/19594, WO 93/19749, WO 93/19750, WO 93/19751, WO 98/18796, WO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607 and WO 04/037805; A_{2A} agonists such as those described in EP 1052264, EP 1241176, EP 409595A2, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462, and WO 03/086408; and A2B antagonists such as those described in WO 02/42298.

Suitable bronchodilatory drugs include anticholinergic or antimuscarinic compounds, in particular ipratropium bromide, oxitropium bromide, tiotropium salts and CHF 4226 (Chiesi), and glycopyrrolate, but also those described in EP 424021, U.S. Pat. No. 3,714,357, U.S. Pat. No. 5,171,744, WO 01/04118, WO 02/00652, WO 02/51841, WO 02/53564, WO 03/00840, WO 03/33495, WO 03/53966, WO 03/87094, WO 04/018422 and WO 04/05285; and beta-2 adrenoceptor agonists such as albuterol (salbutamol), metaproterenol, terbutaline, salmeterol fenoterol, procaterol, and especially, formoterol, carmoterol and pharmaceutically acceptable salts thereof, and compounds (in free or salt or solvate form) of formula (I) of WO 00/75114, which document is incorporated herein by reference, preferably compounds of the Examples thereof, especially a compound of formula

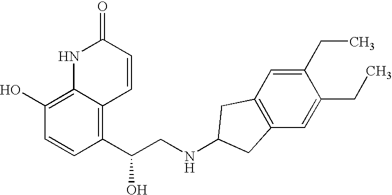

and pharmaceutically acceptable salts thereof, as well as compounds (in free or salt or solvate form) of formula I of WO 04/16601, and also compounds of EP 1440966, JP 05025045, WO 93/18007, WO 99/64035, US 2002/0055651, WO 01/42193, WO 01/83462, WO 02/66422, WO 02/70490, WO 02/76933, WO 03/24439, WO 03/42160, WO 03/42164, WO 03/72539, WO 03/91204, WO 03/99764, WO 04/16578, WO 04/22547, WO 04/32921, WO 04/33412, WO 04/37768, WO 04/37773, WO 04/37807, WO 04/39762, WO 04/39766, WO 04/45618 WO 04/46083 and WO 04/80964.

Such antihistamine drug substances include cetirizine hydrochloride, acetaminophen, clemastine fumarate, promethazine, loratidine, desloratidine, diphenhydramine and fexofenadine hydrochloride.

Combinations of compounds of the invention and anticholinergic or antimuscarinic compounds, steroids, beta-2 agonists, PDE4 inhibitors, dopamine receptor agonists, LTD4 antagonists or LTB4 antagonists may also be used. Other useful combinations of compounds of the invention with anti-inflammatory drugs are those with other antagonists of chemokine receptors, e.g. CCR-1, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, Takeda antagonists such as NI-[[4-[([6,7-dihydro-2-(4-methylphenyl)-5H-benzocyclohepten-8-yl]carbonyliamino]phenyl]-methyl]-tetrahydro-N,N-dimethyl-2H-pyran-4-aminium chloride (TAK-770), CCR-5 antagonists described in U.S. Pat. No. 6,166,037 (particularly claims 18 and 19), WO 0066558 (particularly claim 8), and WO 0066559 (particularly claim 9).

In accordance with the foregoing, the invention also provides a method for the treatment of a condition or disease mediated by CXCR2, for example an inflammatory or allergic condition, particularly an inflammatory or obstructive airways disease, which comprises administering to a subject, particularly a human subject, in need thereof an effective amount of a compound of formula (I) in a free or pharmaceutically acceptable salt form as hereinbefore described. In another aspect the invention provides the use of a compound of formula (I), in free or pharmaceutically acceptable salt form, as hereinbefore described for the manufacture of a medicament for the treatment of a condition or disease mediated by CXCR2, for example an inflammatory or allergic condition or disease, particularly an inflammatory or obstructive airways disease.

The compounds of the invention may be administered by any appropriate route, e.g. orally, for example in the form of a tablet or capsule; parenterally, for example intravenously; by inhalation, for example in the treatment of inflammatory or obstructive airways disease; intranasally, for example in the treatment of allergic rhinitis; topically to the skin, for example in the treatment of atopic dermatitis; or rectally, for example in the treatment of inflammatory bowel disease.

In a further aspect, the invention also provides a pharmaceutical composition comprising as active ingredient a compound of formula (I) in free or pharmaceutically acceptable salt form, optionally together with a pharmaceutically acceptable diluent or carrier therefor. The composition may contain a co-therapeutic compound such as an anti-inflammatory bronchodilatory or antihistamine drug as hereinbefore described. Such compositions may be prepared using conventional diluents or excipients and techniques known in the galenic art. Thus oral dosage forms may include tablets and capsules. Formulations for topical administration may take the form of creams, ointments, gels or transdermal delivery systems, e.g. patches. Compositions for inhalation may comprise aerosol or other atomizable formulations or dry powder formulations.

When the composition comprises an aerosol formulation, it preferably contains, for example, a hydro-fluoro-alkane (HFA) propellant such as HFA134a or HFA227 or a mixture of these, and may contain one or more co-solvents known in the art such as ethanol (up to 20% by weight), and/or one or more surfactants such as oleic acid or sorbitan trioleate, and/or one or more bulking agents such as lactose. When the composition comprises a dry powder formulation, it preferably contains, for example, the compound of formula I having a particle diameter up to 10 microns, optionally together with a diluent or carrier, such as lactose, of the desired particle size distribution and a compound that helps to protect against product performance deterioration due to moisture, e.g. magnesium stearate. When the composition comprises a nebulised formulation, it preferably contains, for example, the compound of formula I either dissolved, or suspended, in a vehicle containing water, a co-solvent such as ethanol or propylene glycol and a stabiliser, which may be a surfactant.

The invention includes (A) a compound of the invention in inhalable form, e.g. in an aerosol or other atomisable composition or in inhalable particulate, e.g. micronised form, (B) an inhalable medicament comprising a compound of the invention in inhalable form; (C) a pharmaceutical product comprising such a compound of the invention in inhalable form in association with an inhalation device; and (D) an inhalation device containing a compound of the invention in inhalable form.

Dosages of compounds of the invention employed in practising the present invention will of course vary depending, for example, on the particular condition to be treated, the effect desired and the mode of administration. In general, suitable daily dosages for administration by inhalation are of the order of 0.01 to 1 mg/kg per day while for oral administration suitable daily doses are of the order of 0.005 to 100 mg/kg of total body weight. The daily parenteral dosage regimen about 0.001 to about 80 mg/kg of total body weight. The daily topical dosage regimen will preferably be from 0.1 mg to 150 mg, administered one to four, preferably two or three times daily.

In the following examples all temperatures are in degree Celsius (°).

### General Conditions for Characterization Data of Exemplified Compounds:

Mass spectra are run on an open access Waters 600/ZQ HPLC/Mass Spectrometer system using electrospray ionization. [M+H]⁺ refers to mono-isotopic molecular weights.

The following ABBREVIATIONS are used:
AcOH acetic acid
DCM dichloromethane
DMF N,N-dimethylformamide
EtOAc ethyl acetate
EtOH ethanol
Et₂O diethylether
HATU [Dimethylamino-([1,2,3]triazolo[4,5-n]pyridin-3-yloxy)-methylene]-dimethyl-ammonium; hexafluorophosphate
MeCN acetonitrile
MeOH methanol
NH₄OAc ammonium acetate
PCC pyridium chlorochromate
RT room temperature

### EXAMPLES

### Example 1

### 2-(2,3-Difluoro-benzylsulfanyl)-4-hydroxy-6-(4-methoxy-phenyl)-pyrimidine-5-carbonitrile

### a) 2-Cyano-3-(4-methoxyphenyl)-acrylic acid ethyl ester

1.34 ml of 4-methoxybenzaldehyde, 0.771 g of NH₄OAc, 0.515 ml of AcOH and 1.08 ml of ethyl-cyanoacetate are dissolved in anhydrous toluene and 4 A molecular sieves are added. The mixture obtained is heated at reflux for 3.5 hours under an argon atmosphere. On cooling, the mixture obtained is filtered to remove the molecular sieves, the filtrate obtained is washed with H₂O/saturated brine and dried. The product obtained is filtrated, solvent is evaporated and the evaporation residue obtained is redissolved in DCM. Solvent is evaporated and the residue obtained is dried.

2-Cyano-3-(4-methoxyphenyl)-acrylic acid ethyl ester is obtained.

### b) 2-(2,3-Difluorobenzyl)-isothiourea hydrobromide

A suspension of 40.33 g of 2,3-difluorobenzyl bromide and 17.8 g of thiourea in 240 ml of EtOH is heated at 65° for 3.5 hours. The reaction mixture obtained is cooled and solvent is evaporated. The evaporation residue obtained is triturated with ether. A solid obtained is filtered, washed with Et₂O and dried.

2-(2,3-Difluorobenzyl)-isothiourea hydrobromide is obtained.

c) 2.17 g of 2-Cyano-3-(4-methoxyphenyl)-acrylic acid ethyl ester, 2.53 g of 2-(2,3-difluorobenzyl)-isothiourea hydrobromide and 4.3 ml of N,N-diisopropylethylamine are dissolved in 30 ml of EtOH. The reaction mixture obtained is stirred for 66 hours at RT and solvent is evaporated. The evaporation residue obtained is dissolved in EtOAc and washed with H₂O, 10% citric acid and saturated brine. A solid formed is filtered off, the filtrate obtained is washed with EtOAc and dried. 2-(2,3-difluorobenzylsulfanyl)-4-hydroxy-6-(4-methoxyphenyl)-pyrimidine-5-carbonitrile is obtained.

### Example 2

### 4-(3,4-Dichlorophenyl)-2-(2,3-difluorobenzylsulfanyl-6-hydroxy-pyrimidine-5-carbonitrile

### a) 2-Cyano-3-(3,4-dichlorophenyl)-acrylic acid ethyl ester

1.93 g of 3,4-dichlorobenzaldehyde, 0.771 g of NH₄OAc, 0.515 ml of AcOH and 1.08 ml of ethyl cyanoacetate are dissolved in 10 ml of anhydrous toluene. 4 A molecular sieves are added and the reaction mixture obtained is heated at reflux for 3.5 hours under an argon atmosphere. On cooling, the mixture obtained is filtered to remove the molecular sieves and the solid which precipitates out. The solid obtained is washed with toluene. The filtrate obtained is washed with H₂O and saturated brine and dried. After filtration solvent is evaporated and the evaporation residue obtained is dissolved in DCM. Solvent is evaporated, the evaporation residue obtained is dried.

2-Cyano-3-(3,4-dichlorophenyl)-acrylic acid ethyl ester is obtained.

b) 2.422 g of 2-Cyano-3-(3,4-dichlorophenyl)-acrylic acid ethyl ester and 2.42 g of a compound of Example 1b) are dissolved in 30 ml of EtOH together with 4.1 ml of N,N-diisopropylethylamine. The reaction mixture obtained is stirred for about 60 hours at RT. Solvent is evaporated, the evaporation residue obtained is dissolved in EtOAc and washed with H₂O, 10% citric acid and saturated brine and dried. The mixture obtained is filtrated and solvent is evaporated. The evaporation residue obtained is dissolved in DCM and after evaporation and trituration from EtOAc, a solid is filtered off, the filtrate obtained is washed with EtOAc and dried. 4-(3,4-Dichlorophenyl)-2-(2,3-difluorobenzylsulfanyl-6-hydroxy-pyrimidine-5-carbonitrile is obtained.

### Example 3

### 2-(2,3-Difluorobenzylsulfanyl)-4-hydroxy-6-(4-hydroxyphenyl)-pyrimidine-5-carbonitrile

### a) 2-Cyano-3-(4-hydroxy-phenyl)-acrylic acid ethyl ester

This compound is prepared analogously to the compound of Example 2a but using the appropriate starting materials.

b) 2.39 g of 2-Cyano-3-(4-hydroxy-phenyl)-acrylic acid ethyl ester and 5.1 ml of a compound of Example 1b) are dissolved in 30 ml of EtOH together with 5.1 ml of N,N-diisopropylethylamine. The reaction mixture obtained is stirred for about 60 hours at RT. Solvent is evaporated and the residue obtained is redissolved in EtOAc, washed with H₂O, 10% citric acid and saturated brine and dried. The mixture obtained is filtrated, solvent is evaporated and the residue obtained is further purified. 2-(2,3-Difluorobenzyl-sulfanyl)-4-hydroxy-6-(4-hydroxyphenyl)-pyrimidine-5-carbonitrile is obtained.

### Example 4

### 4-cyclopropyl-2-(2,3-difluorobenzylsulfanyl-6-hydroxypyrimidine-5-carbonitrile

### a) (Z)-2-Cyano-3-cyclopropyl-acrylic acid ethyl ester

0.82 ml of Cyclopropane carboxaldehyde, 0.771 g of NH₄OAc, 0.515 ml of AcOH and 1.08 ml of ethyl cyanoacetate are dissolved in 10 ml of anhydrous toluene. 4 Å molecular sieves are added and the mixture obtained is heated at reflux for about 5 hours under an argon atmosphere. On cooling, the mixture obtained is filtered to remove the molecular sieves, the filtrate obtained is washed with H₂O and saturated brine and dried. After filtration, solvent is evaporated and the residue obtained is dried.

(Z)-2-Cyano-3-cyclopropyl-acrylic acid ethyl ester is obtained.

b) 1.65 g of (Z)-2-Cyano-3-cyclopropyl-acrylic acid ethyl ester and 2.70 g of a compound of Example 1b) are dissolved in 30 ml of EtOH together with 4.60 ml of N,N-diisopropyl-ethylamine. The reaction mixture obtained is stirred for 20 hours at RT. Solvent is evaporated, the residue obtained is redissolved in EtOAC and is washed with H₂O, 10% citric acid and saturated brine. A precipitate obtained is filtered, the filtrate obtained is washed with EtOAc and dried. 4-cyclopropyl-2-(2,3-difluorobenzylsulfanyl-6-hydroxypyrimidine-5-carbonitrile is obtained.

The following compounds 5 to 24 are obtained according to the procedure described above and using the appropriate starting materials. Compounds of examples 1 to 24 are summarized in Table 1 below.

TABLE 1

| EX. | Structure | [M + H]⁺ |
|---|---|---|
| 1 | 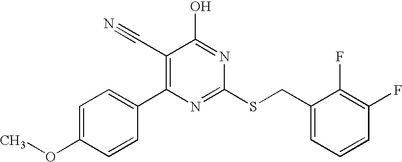 | 385.52 |
| 2 | 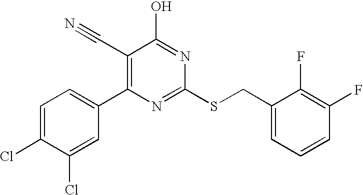 | 423.37 & 425.38 |
| 3 | 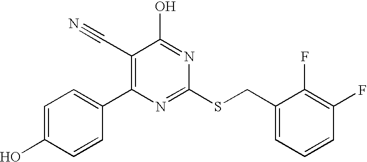 | 372.05 |
| 4 | 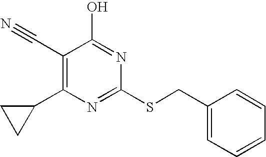 | 284.09 |
| 5 | 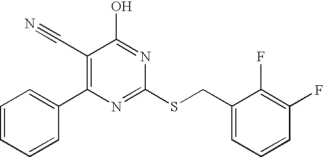 | 356 |
| 6 | 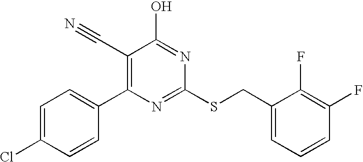 | 389.46 & 391.50 |
| 7 | 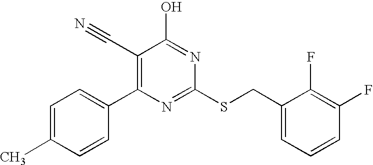 | 369.54 & 370.59 |
| 8 | 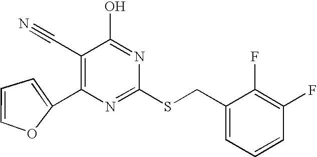 | 346.64 |
| 9 | 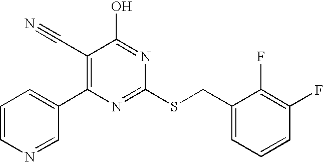 | 356.49 |
| 10 | 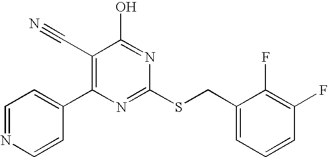 | 356.49 |
| 11 | 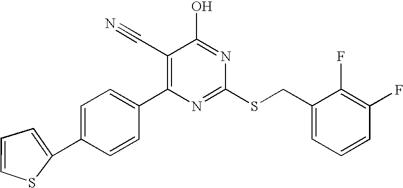 | 361.46 |
| 12 | 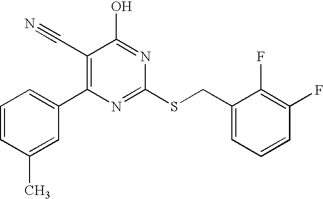 | 370.06 |
| 13 | 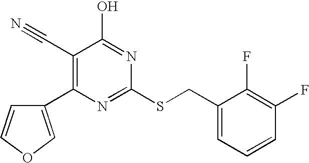 | 346.01 |
| 14 | 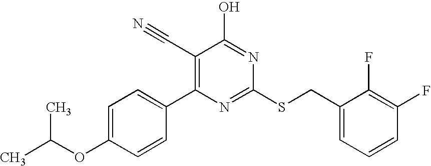 | 414.16 |
| 15 | 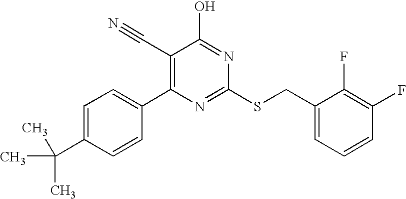 | |
| 16 | 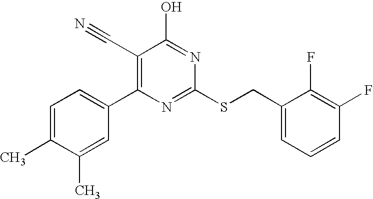 | 384.09 |
| 17 | 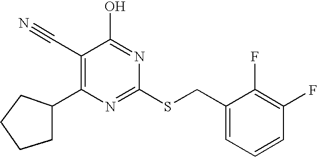 | 348.07 |
| 18 | 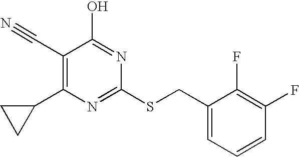 | 320.06 |
| 19 | 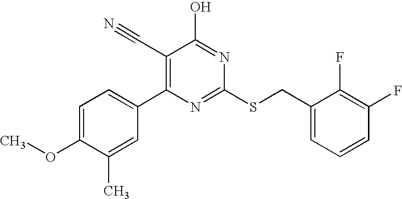 | 400.12 |
| 20 | 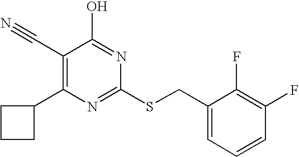 | 334.04 |
| 21 | 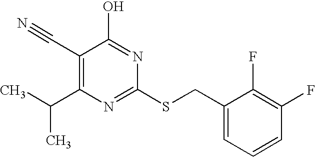 | 322.08 |
| 22 | 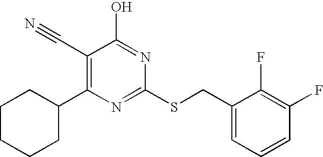 | 362.09 |
| 23 | 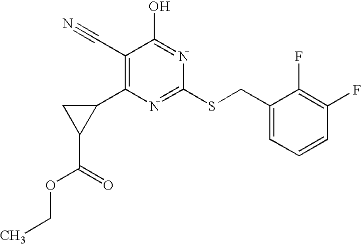 | 392.12 |
| 24 | 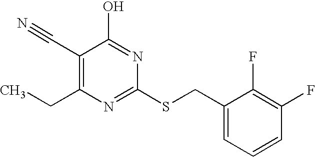 | 308.08 |

Further compounds of the present invention include compounds of formula (I) wherein R₁ is 2,3-difluorobenzyl and R₂ is shown in Table 2 below, the method of preparation being described hereinafter. The table also shows mass spectrometry data.

TABLE 2

| EX. | R₂ | Name | [M + H]⁺ |
|---|---|---|---|
| 25 | 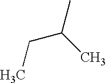 | 4-sec-Butyl-2-(2,3-difluoro benzyl sulfanyl)-6- hydroxy-pyrimidine-5-carbonitrile | 336 |
| 26 | 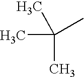 | 4-tert-Butyl-2-(2,3-difluoro-benzylsulfanyl)-6- hydroxy-pyrimidine-5-carbonitrile | 336 |
| 27 | 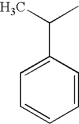 | 2-(2,3-Difluoro-benzylsulfanyl)-4-hydroxy-6-(1- phenyl-ethyl)-pyrimidine-5-carbonitrile | 384 |
| 28 | 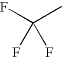 | 2-(2,3-Difluoro-benzylsulfanyl)-4-hydroxy-6- trifluoromethyl-pyrimidine-5-carbonitrile | no Mass Ion seen |
| 29 | 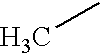 | 2-(2,3-Difluoro-benzylsulfanyl)-4-hydroxy-6- methyl-pyrimidine-5-carbonitrile | 294 |
| 30 | 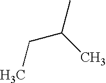 | 4-sec-Butyl-2-(2,3-difluoro-benzyl sulfanyl)-6- hydroxy pyrimidine-5-carbonitrile | 336 |
| 31 | 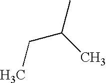 | 4-sec-Butyl-2-(2,3-difluoro-benzyl sulfanyl)-6- hydroxy pyrimidine-5-carbonitrile | 336 |
| 32 | 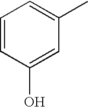 | 2-(2,3-Difluoro-benzylsulfanyl)-4-hydroxy-6-(3- hydroxy-phenyl)-pyrimidine-5-carbonitrile | 401 |
| 33 | 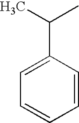 | 2-(2,3-Difluoro-benzylsulfanyl)-4-hydroxy-6-(1- phenyl-ethyl)-pyrimidine-5-carbonitrile | 384 |
| 34 | 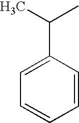 | 2-(2,3-Difluoro-benzylsulfanyl)-4-hydroxy-6-(1- phenyl-ethyl)-pyrimidine-5-carbonitrile | 384 |
| 35 | 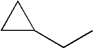 | 4-Cyclopropylmethyl-2-(2,3-difluoro- benzylsulfanyl)-6 hydroxy-pyrimidine-5- carbonitrile | 334 |
| 36 | 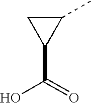 | Trans-2-[5-Cyano-2-(2,3-difluoro- benzylsulfanyl)-6 hydroxy-pyrimidin-4- yl]cyclopropane carboxylic acid | 364 |
| 37 | 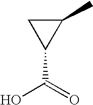 | (1R,2R)-2-[5-Cyano-2-(2,3-difluoro- benzylsulfanyl)-6 hydroxy-pyrimidin-4- yl]cyclopropane carboxylic acid | 364 |
| 38 | 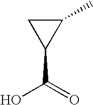 | (1S,2S)-2-[5-Cyano-2-(2,3-difluoro- benzylsulfanyl)-6 hydroxy-pyrimidin-4- yl]cyclopropane carboxylic acid | 364 |
| 39 | 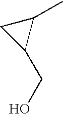 | Trans-2-(2,3-Difluoro-benzylsulfanyl)-4- hydroxy-6-(2-hydroxymethyl-cyclopropyl)- pyrimidine-5-carbonitrile | 350 |
| 40 | 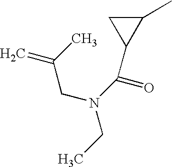 | Trans-2-[5-Cyano-2-(2,3-difluoro-benzyl sulfanyl)-6-hydroxy-pyrimidin-4-yl]cyclopropane carboxylic acid ethyl-(2-methyl-allyl)-amide | 445 |
| 41 | 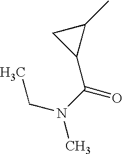 | 2-[5-Cyano-2-(2,3-difluoro-benzylsulfanyl)-6- hydroxy-pyrimidin-4-yl]-cyclopropane carboxylic acid ethyl-methyl-amide | 405 |
| 42 | 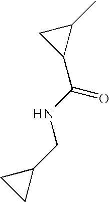 | 2-[5-Cyano-2-(2,3-difluoro-benzylsulfanyl)-6- hydroxy-pyrimidin-4-yl]-cyclopropane carboxylic acid cyclopropyl methyl-amide | 417 |
| 43 | 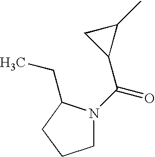 | 2-(2,3-Difluoro-benzylsulfanyl)-4-[2-(2-ethyl- pyrrolidine-1carbonyl)-cyclopropyl]-6- hydroxypyrimidine-5-carbonitrile | 445 |
| 44 | 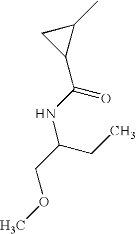 | 2-[5-Cyano-2-(2,3-difluoro-benzylsulfanyl)-6- hydroxy-pyrimidin-4-yl]-cyclopropane carboxylic acid (1-methoxymethyl-propyl)- amide | 449 |
| 45 | 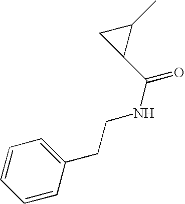 | 2-[5-Cyano-2-(2,3-difluoro-benzylsulfanyl)-6- hydroxy-pyrimidin-4-yl]-cyclopropane carboxylic acid phenethyl-amide | 467 |
| 46 | 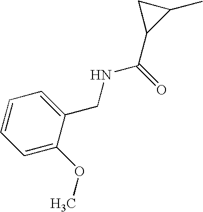 | 2-[5-Cyano-2-(2,3-difluoro-benzylsulfanyl)-6- hydroxy-pyrimidin-4-yl]-cyclopropane carboxylic acid 2-methoxy-benzylamide | 483 |
| 47 | 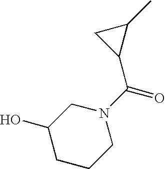 | 2-(2,3-Difluoro-benzylsulfanyl)-4-hydroxy-6-[2- (3-hydroxy-piperidine-1-carbonyl)cyclopropyl]- pyrimidine-5-carbonitrile | 447 |
| 48 | 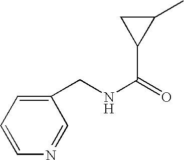 | 2-[5-Cyano-2-(2,3-difluoro-benzylsulfanyl)-6- hydroxy-pyrimidin-4-yl]-cyclopropane carboxylic acid (pyridin-3-yl methyl)-amide | 454 |
| 49 | 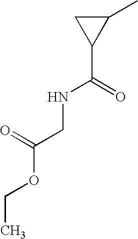 | Trans-({2-[5-Cyano-2-(2,3-difluoro- benzylsulfanyl)-6-hydroxy-pyrimidin-4-yl]- cyclopropane carbonyl}-amino)-acetic acid ethyl ester | 449 |
| 50 | 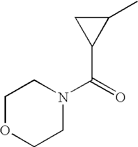 | 2-(2,3-Difluoro-benzylsulfanyl)-4-hydroxy-6-[2- (morpholine-4-carbonyl)- cyclopropyl]pyrimidine-5-carbonitrile | 433 |
| 51 | 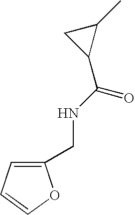 | 2-[5-Cyano-2-(2,3-difluoro-benzylsulfanyl)-6- hydroxy-pyrimidin-4-yl]-cyclopropane carboxylic acid(furan-2-ylmethyl)-amide | 443 |
| 52 | 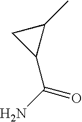 | 2-[5-Cyano-2-(2,3-difluoro-benzylsulfanyl)-6- hydroxy-pyrimidin-4-yl]-cyclopropane carboxylic acid amide | 363 |
| 53 | 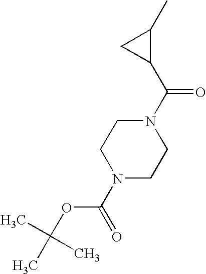 | Trans-4-{2-[5-Cyano-2-(2,3-difluoro- benzylsulfanyl)-6-hydroxy-pyrimidin-4-yl]- cyclopropane carbonyl}-piperazine-1- carboxylic acid tert-butyl ester | 1063 (dimer) |
| 54 | 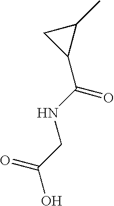 | Trans-({2-[5-Cyano-2-(2,3-difluoro- benzylsulfanyl)-6-hydroxy-pyrimidin-4-yl]- cyclopropane carbonyl}-amino)-acetic acid | 421 |
| 55 | 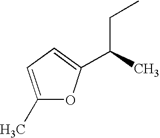 | 2-(2,3-Difluoro-benzylsulfanyl)-4-hydroxy-6- [(R)-2-(5-methyl-furan-2-yl)-propyl]-pyrimidine- 5-carbonitrile | 402 |
| 56 | 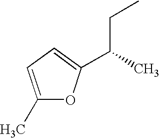 | 2-(2,3-Difluoro-benzylsulfanyl)-4-hydroxy-6- [(S)-2-(5-methyl-furan-2-yl)-propyl]-pyrimidine- 5-carbonitrile | 402 |
| 57 | 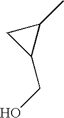 | (1S,2S)-2-(2,3-Difluoro-benzylsulfanyl)-4- hydroxy-6-(2-hydroxymethyl-cyclopropyl)- pyrimidine-5-carbonitrile | 350 |
| 58 | 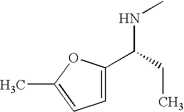 | 2-(2,3-Difluoro-benzylsulfanyl)-4-hydroxy-6- [(R)-1-(5-methyl-furan-2-yl)-propylamino]- pyrimidine-5-carbonitrile | 417 |
| 59 | 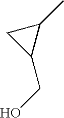 | (1R,2R)-2-(2,3-Difluoro-benzylsulfanyl)-4- hydroxy-6-(2-hydroxymethyl-cyclopropyl)- pyrimidine-5-carbonitrile | 350 |

### Example 25

### 4-sec-Butyl-2-(2,3-difluoro benzyl sulfanyl)-6-hydroxy-pyrimidine-5-carbonitrile

1.050 ml of 2-Methylbutylaldehyde, 1.083 ml of ethylcyanoacetate, 1.38 g of K₂CO₃, 1.05 ml of 2-(2,3-difluoro-benzyl)-isothiourea hydrobromide (Intermediate A) and 20 ml of EtOH are mixed and heated to 60° for 3.25 hours. The reaction mixture obtained is cooled to RT and left to stand overnight. The inorganic solids are removed by filtration, solvent from the filtration residue obtained is evaporated and the evaporation residue obtained is dissolved in EtOAc. The organic portion obtained is washed with H₂O and brine, dried, filtered and concentrated. The crude residue is dry loaded onto silica and purification by flash chromatography on silica, eluting with EtOAc:iso-hexanes (1:1). The title compound is obtained.

### Examples 26-35

These compounds are prepared analogously to Example 25 by using the appropriate starting materials. The compounds are recovered from reaction mixtures and purified using conventional techniques such as, for example, flash chromatography, reverse phase chromatography or chiral HPLC purification for racemates to give the two enantiomers.

### Example 36

### Trans-2-[5-cyano-2-(2,3-difluoro-benzylsulfanyl)-6 hydroxy-pyrimidin-4-yl]cyclo-propane carboxylic acid

177 mg of Trans-2-[5-cyano-2-(2,3-difluoro-benzylsulfanyl)-6-hydroxy-pyrimidin-4-yl]-cyclopropanecarboxylic acid ethyl ester (Intermediate B) are suspended in 5 ml of EtOH. 2.7 ml of 1M NaOH are added and the reaction mixture obtained is stirred at RT for 20 hours. Solvent is evaporated and the evaporation residue obtained is dissolved in H₂O and washed with Et₂O. The aqueous layer obtained is acidified with 1M HCl, a precipitate formed is collected by filtration, the filtration residue obtained is washed with H₂O and Et₂O and dried. The title compound is obtained.

### Examples 37 and 38

### (1R,2R)-2-[5-Cyano-2-(2,3-difluoro-benzylsulfanyl)-6 hydroxy-pyrimidin-4-yl]cyclopropane carboxylic acid and (1S,2S)-2-[5-Cyano-2-(2,3-difluoro-benzylsulfanyl)-6 hydroxy-pyrimidin-4-yl]cyclopropane carboxylic acid

The compounds are isolated from the purification of Trans-2-[5-cyano-2-(2,3-difluoro-benzylsulfanyl)-6 hydroxy-pyrimidin-4-yl]cyclopropane carboxylic acid (Example 36) by chiral HPLC purification (95% hexane/5% ethanol/0.1% TFA Column Chiralcel OD 2 cm×25 cm lambda=220 nm) to afford the title compounds as the single enantiomers.

### Example 39

### Trans-2-(2,3-Difluoro-benzylsulfanyl)-4-hydroxy-6-(2-hydroxymethyl-cyclopropyl)-pyrimidine-5-carbonitrile

410 mg of a compound of example 36 are suspended in 5 ml of dry THF under an atmosphere of argon. The reaction mixture obtained is cooled to −5° (dry-ice/acetone) and 2.1 ml of borane in THF are added slowly over a period of 10 minutes. The reaction mixture obtained is warmed to RT overnight and stirred for a further 3.5 hours. The reaction mixture obtained is carefully quenched with 10 ml of H₂O and 290 mg of K₂CO₃ are added. The mixture obtained is washed 2× with Et₂O and the aqueous portion obtained is concentrated. The crude residue obtained is dry loaded onto silica and purification by flash chromatography on silica eluting with EtOAc and then EtOH:EtOAC (1:9). The title compound is obtained.

### Example 40

### Trans-2-[5-Cyano-2-(2,3-difluoro-benzyl sulfanyl)-6-hydroxy-pyrimidin-4-yl]cyclopropane carboxylic acid ethyl-(2-methyl-allyl)-amide

100 mg of a compound of example 36 are dissolved in 3 ml of DMF and 0.078 ml of triethylamine and 97 mg of HATU are added. The reaction mixture obtained is stirred at RT for 25 minutes. 0.041 ml of N-Ethyl-2-methylallylamine are added and the reaction mixture obtained is continued stirring overnight. The reaction mixture obtained is concentrated and the residue obtained is dissolved in DCM. The organic portion obtained is washed with 10% citric acid and brine, dried, filtered and concentrated. The title compound is obtained.

### Examples 41 to 53

### These compounds are prepared analogously to Example 40 by using the appropriate starting materials

### Example 54

### Trans-({2-[5-Cyano-2-(2,3-difluoro-benzylsulfanyl)-6-hydroxy-pyrimidin-4-yl]-cyclopropane carbonyl}-amino)-acetic acid

This compound is prepared analogously to Example 36 by using the appropriate starting materials.

### Examples 55 and 56

### 2-(2,3-Difluoro-benzylsulfanyl)-4-hydroxy-6-[(R)-2-(5-methyl-furan-2-yl)-propyl]-pyrimidine-5-carbonitrile and 2-(2,3-Difluoro-benzylsulfanyl)-4-hydroxy-6-[(S)-2-(5-methyl-furan-2-yl)-propyl]-pyrimidine-5-carbonitrile

These compounds are prepared analogously to Example 49 by using the appropriate starting materials to afford the crude products as the trans racemate. Purification of the racemate by chiral HPLC separation (95% hexane/5% ethanol/0.1% TFA Column Chiralcel OD 2 cm×25 cm lambda=220 nm) affords the title compounds as single enantiomers.

### Examples 57 and 58

### 2-(1S,2S)-(2,3-Difluoro-benzylsulfanyl)-4-hydroxy-6-(2-hydroxymethyl-cyclopropyl)-pyrimidine-5-carbonitrile and 2-(1R,2R)-(2,3-Difluoro-benzylsulfanyl)-4-hydroxy-6-(2-hydroxymethyl-cyclopropyl)-pyrimidine-5-carbonitrile

These compounds are prepared analogously to Example 39 from the enantiomeric starting acids, Examples 27 and 28. The final products are purified by chiral HPLC separation.

### Example 59

### 2-(2,3-Difluoro-benzylsulfanyl)-4-hydroxy-6-[(R)-1-(5-methyl-furan-2-yl)-propylamino]-pyrimidine-5-carbonitrile

### Step 1: 4-Chloro-2-(2,3-difluoro-benzylsulfanyl)-6-[(R)-1-(5-methyl-furan-2-yl)-propylamino]-pyrimidine-5-carbonitrile

200 mg of Intermediate D, 180 mg of (R)-1-(5-Methyl-furan-2-yl)-propylamine (prepared as described in WO03/080053 and WO03/057676), 180 mg of K₂CO₃ and 3 ml of EtOH are stirred at RT for 24 hours. The reaction mixture obtained is absorbed onto silica and purification by flash chromatography on silica eluting with iso-hexanes:EtOAc (20:80) affords the title compound.

### Step 2: 2-(2,3-Difluoro-benzylsulfanyl)-4-hydroxy-6-[(R)-1-(5-methyl-furan-2-yl)-propylamino]-pyrimidine-5-carbonitrile

160 mg of 4-Chloro-2-(2,3-difluoro-benzylsulfanyl)-6-[(R)-1-(5-methyl-furan-2-yl)-propylamino]-pyrimidine-5-carbonitrile, 50 mg of potassium tert-butoxide and 3 ml of dry toluene are stirred at 120° for 24 hours under an inert atmosphere of argon. After cooling to RT, H₂O is added, the mixture obtained is stirred for 20 minutes and then acidified with AcOH to pH4. A precipitate formed s filtered, the filtrate obtained is absorbed onto silica and purification by flash chromatography on silica eluting with iso-hexanes:EtOAc (20% to 3%) affords the title compound.

### Preparation Of Intermediates

### Intermediate A: 2-(2,3-Difluoro-benzyl)-isothiourea hydrobromide

53.3 g of 2,3-difluorobenzyl bromide and 23.5 g of thiourea are suspended in 310 ml of EtOH and the reaction mixture obtained is heated to 65° for 3.5 hours. After cooling to RT, solvent is evaporated and the residue obtained is triturated with EtOAc. The solid obtained is collected by filtration and dried. The title compound is obtained.

### Intermediate B: Trans-2-[5-Cyano-2-(2,3-difluoro-benzylsulfanyl)-6-hydroxy-pyrimidin-4-yl]-cyclopropanecarboxylic acid ethyl ester

This compound is made analogously to Example 29 by replacing 2-methyl butylaldehyde with ethyl 2-formyl-1-cyclopropanecarboxylate and by replacing water in the work-up with 10% citric acid to afford the title compound.

### Intermediate C: Cyclopropylacetaldehyde

2.33 g of Cyclopropylethanol are dissolved in 100 ml of DCM and 7.58 g of PCC are added. The reaction mixture obtained is stirred at RT for 24 hours. The reaction mixture obtained is filtered through Celite® (filter agent) and silica (50:50 by volume) washing with DCM (750 ml). The organic solvent is reduced in vacuo to 20 ml volume and used without isolation or further purification.

### Intermediate D: 4,6-Dichloro-2-(2,3-difluoro-benzylsulfanyl)-pyrimidine-5-carbonitrile

### Step 1: 2-(2,3-Difluoro-benzylsulfanyl)-pyrimidine-4,6-diol

To a suspension of 15.2 g of 2-thiobarbituric acid in 85 ml of EtOH and 85 ml of H₂O are added a solution of 4.2 g of NaOH in 25 ml of EtOH and 25 ml of H₂O. 21.74 g of Difluorobenzyl bromide are added dropwise and the reaction mixture obtained is heated at 60° for 2 hours and then stirred at RT overnight. A precipitate formed is collected by filtration, washed with 200 ml of H₂O and 20 ml of iso-propanol and dried. The title compound is obtained.

### Step 2: 4,6-Dichloro-2-(2,3-difluoro-benzylsulfanyl)-pyrimidine-5-carbaldehyde

32.8 ml of POCl₃ are treated with the dropwise addition of 10.67 ml of dry at 5° (ice-bath), under an inert atmosphere of argon maintaining the temperature at 5°. The reaction mixture obtained is warmed to RT and stirred for 2 hours. 25.5 g of 2-(2,3-Difluoro-benzylsulfanyl)-pyrimidine-4,6-diol are added portionwise and the reaction mixture obtained is stirred at RT for 1 hour then heated at 100° for 17 hours. After cooling to RT the reaction mixture obtained is carefully poured onto 2.2 l of ice-water with overhead (mechanical) stirring for 20 minutes. 1 l of EtOAc is added, the layers formed are separated and the aqueous portion obtained is extracted 2× with 600 ml of EtOAc. The organic portions obtained are combined, dried, filtered and concentrated. The crude residue obtained may be absorbed onto silica and purification by flash chromatography on silica eluting with EtOAc:iso-hexanes (5:95). The title compound is obtained.

### Step 3: 4,6-Dichloro-2-(2,3-difluoro-benzylsulfanyl)-pyrimidine-5-carbaldehydeoxime

A suspension of 21.39 g of 4,6-dichloro-2-(2,3-difluoro-benzylsulfanyl)-pyrimidine-5-carbaldehyde 5.4 ml of H₂O and 84 ml of AcOH is treated with 4.88 g of hydroxylamine hydrochloride. The reaction mixture obtained is heated at 60° for 3 hours, allowed to cool to RT and 85 ml of H₂O are added. A precipitate formed is cooled in ice for 1 hour and collected by filtration and dried in a vacuum oven (45°) overnight. The crude residue obtained is absorbed onto silica and purification by flash chromatography on silica and eluting with DCM affords the title compound.

### Step 4: 4,6-Dichloro-2-(2,3-difluoro-benzylsulfanyl)-pyrimidine-5-carbonitrile

750 mg of 4,6-Dichloro-2-(2,3-difluoro-benzylsulfanyl)-pyrimidine-5-carbaldehydeoxime and 2 ml of thionyl chloride are mixed together and heated at 40° for 3 hours. After cooling to RT, thionyl chloride is removed and the residue obtained is stirred in ice-cold H₂O. A precipitate formed is collected by filtration and the filtration residue obtained is dried.

The title compound is obtained.

1. A method for the prevention or treatment of a CXCR2 receptor mediated condition or disease comprising administering an effective amount of at least one compound of formula 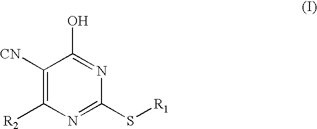
wherein
R₁ is (C₆₋₁₈)aryl or (C₆₋₁₈)aryl(C₁₋₄)alkyl, unsubstituted or one- or morefold substituted by (C₁₋₄)alkyl, C₁₋₄)alkoxy, hydroxy, halogen,
R₂ is
(C₁₋₈)alkyl, halo(C₁₋₈)alkyl, amino, substituted amino,
(C₆₋₁₈)aryl, unsubstituted or one- or morefold substituted by (C₁₋₄)alkyl, C₁₋₄)alkoxy, hydroxy or halogen,
(C₆₋₁₈)aryl(C₁₋₈)alkyl, unsubstituted or one- or morefold substituted,
heterocyclyl or heterocyclyl(C₁₋₈)alkyl, wherein heterocyclyl has 5 to 6 ring members and 1 to 4 heteroatoms selected from N, O, S, unsubstituted or one- or morefold substituted,
(C₃₋₈)cycloalkyl or (C₃₋₈)cycloalkyl(C₁₋₈)alkyl, unsubstituted or, one- or morefold substituted,
or a pharmaceutically acceptable salt or solvate thereof.

2. The method of claim 1, wherein
R₁ is phenyl or phenyl(C₁₋₂)alkyl, unsubstituted or one- or morefold substituted by (C₁₋₄)alkyl, C₁₋₄)alkoxy, hydroxy, halogen,
R₂ is
(C₁₋₄)alkyl, halo(C₁₋₄)alkyl, amino and substituted amino,
unsubstituted phenyl or phenyl substituted by (C₁₋₄)alkyl, C₁₋₄)alkoxy, hydroxy, halogen, heterocyclyl,
phenyl(C₁₋₄)alkyl or phenyl(C₁₋₄)alkyl, unsubstituted or one- or twofold substituted,
heterocyclyl or heterocyclyl(C₁₋₄)alkyl, wherein heterocyclyl has 5 to 6 ring members and 1 to 4 heteroatoms selected from N, O, S, unsubstituted or one- or twofold substituted,
(C₃₋₆)cycloalkyl or (C₃₋₈)cycloalkyl(C₁₋₄)alkyl, unsubstituted or one- or twofold substituted.

3. The method of claim 1, wherein
R₁ is unsubstituted benzyl or benzyl twofold substituted by fluoro,
R₂ is
methyl, sec.butyl, tert.butyl, trifluoromethyl, amino substituted by 2-methylfuran-5-yl-1-propyl,
unsubstituted phenyl or phenyl one or twofold substituted by methyl, methoxy, iso-propoxy, hydroxy, chloro or thienyl,
1-phenyl-ethyl,
furanyl, 5-methyl-furan-2-yl-2-propyl, thienyl or pyridinyl,
unsubstituted cyclopropylmethyl,
unsubstituted cyclopropyl, cyclobutyl, cyclopentyl, cyclohexyl
cyclopropyl onefold substituted by ethoxycarbonyl, hydroxymethyl, hydroxycarbonyl, or a group of formula NR₃R₄—C═O—, wherein
R₃ and R₄ independently are hydrogen, methyl, ethyl, 2-methyl-allyl, cyclopropylmethyl, methoxymethyl, ethoxycarbonylmethyl, hydroxymethyl, hydroxycarbonylmethyl, phenetyl, benzyl, benzyl substituted by methoxy, pyridin-3-yl-methyl, furan-2-yl-methyl, or
R₃ and R₄ together form a 5 membered alicyclic ring system substituted by ethyl, a 6 membered alicyclic ring substituted by hydroxy, a 6 membered alicyclic ring having 0 as a further heteroatom, a 6 membered alicyclic ring having N as a further heteroatom which further N is substituted by tert.-butyloxycarbonyl.

4. The method of claim 1, wherein the compound of formula (I) is administered in combination with at least one second drug substance.

5. The method of claim 1, wherein the CXCR2 receptor mediated condition or disease is an inflammatory or allergic condition or disease.

6. A compound selected from the group consisting of
2-(2,3-Difluoro-benzylsulfanyl)-4-hydroxy-6-(4-methoxy-phenyl)-pyrimidine-5-carbonitrile,
4-(3,4-Dichlorophenyl)-2-(2,3-difluorobenzylsulfanyl-6-hydroxy-pyrimidine-5-carbonitrile,
2-(2,3-Difluorobenzylsulfanyl)-4-hydroxy-6-(4-hydroxyphenyl)-pyrimidine-5-carbonitrile,
4-cyclopropyl-2-(2,3-difluorobenzylsulfanyl-6-hydroxypyrimidine-5-carbonitrile,
2-(2,3-Difluoro-benzylsulfanyl)-4-hydroxy-6-phenyl-pyrimidine-5-carbonitrile,
4-(4-Chlorophenyl)-2-(2,3-difluoro-benzyl-sulfanyl)-6-hydroxy-6-phenyl-pyrimidine-5-carbonitrile,
4-(4-Methylphenyl)-2-(2,3-difluoro-benzylsulfanyl)-6-hydroxy-6-phenyl-pyrimidine-5-carbonitrile,
2-(2,3-Difluoro-benzylsulfanyl)-4-furan-2-yl-6-hydroxy-pyrimidine-5-carbonitrile,
2-(2,3-Difluoro-benzylsulfanyl)-4-hydroxy-6-pyridin-3-yl-pyrimidine-5-carbonitrile,
2-(2,3-Difluoro-benzylsulfanyl)-4-hydroxy-6-pyridin-4-yl-pyrimidine-5-carbonitrile,
2-(2,3-Difluoro-benzylsulfanyl)-4-hydroxy-6-(4-thiophen-2-yl-phenyl)-pyrimidine-5-carbonitrile,
2-(2,3-Difluoro-benzylsulfanyl)-4-hydroxy-6-m-tolyl-pyrimidine-5-carbonitrile,
2-(2,3-Difluoro-benzylsulfanyl)-4-furan-3-yl-6-hydroxy-pyrimidine-5-carbonitrile,
2-(2,3-Difluoro-benzylsulfanyl)-4-hydroxy-6-(4-isopropoxy-phenyl)-pyrimidine-5-carbonitrile,
4-(4-tert-Butyl-phenyl)-2-(2,3-difluoro-benzylsulfanyl)-6-hydroxy-pyrimidine-5-carbonitrile,
2-(2,3-Difluoro-benzylsulfanyl)-4-(3,4-dimethyl-phenyl)-6-hydroxy-pyrimidine-5-carbonitrile,
4-Cyclopentyl-2-(2,3-difluoro-benzylsulfanyl)-6-hydroxy-pyrimidine-5-carbonitrile,
4-Cyclopropyl-2-(2,3-difluoro-benzylsulfanyl)-6-hydroxy-pyrimidine-5-carbonitrile,
2-(2,3-Difluoro-benzylsulfanyl)-4-hydroxy-6-(4-methoxy-3-methyl-phenyl)-pyrimidine-5-carbonitrile,
4-Cyclobutyl-2-(2,3-difluoro-benzylsulfanyl)-6-hydroxy-pyrimidine-5-carbonitrile,
2-(2,3-Difluoro-benzylsulfanyl)-4-hydroxy-6-isopropyl-pyrimidine-5-carbonitrile,
4-Cyclohexyl-2-(2,3-difluoro-benzylsulfanyl)-6-hydroxy-pyrimidine-5-carbonitrile,
2-[5-Cyano-2-(2,3-difluoro-benzylsulfanyl)-6-hydroxy-pyrimidin-4-yl]-cyclopropanecarboxylic acid ethyl ester,
2-(2,3-Difluoro-benzylsulfanyl)-4-ethyl-6-hydroxy-pyrimidine-5-carbonitrile,
4-sec-Butyl-2-(2,3-difluoro benzyl sulfanyl)-6-hydroxy-pyrimidine-5-carbonitrile,
4-tert-Butyl-2-(2,3-difluoro-benzylsulfanyl)-6-hydroxy-pyrimidine-5-carbonitrile,
2-(2,3-Difluoro-benzylsulfanyl)-4-hydroxy-6-(1-phenyl-ethyl)-pyrimidine-5-carbonitrile,
2-(2,3-Difluoro-benzylsulfanyl)-4-hydroxy-6-trifluoromethyl-pyrimidine-5-carbonitrile,
2-(2,3-Difluoro-benzylsulfanyl)-4-hydroxy-6-methyl-pyrimidine-5-carbonitrile,
4-sec-Butyl-2-(2,3-difluoro-benzyl sulfanyl)-6-hydroxy pyrimidine-5-carbonitrile single enantiomer E1,
4-sec-Butyl-2-(2,3-difluoro-benzyl sulfanyl)-6-hydroxy pyrimidine-5-carbonitrile single enantiomer E2,
2-(2,3-Difluoro-benzylsulfanyl)-4-hydroxy-6-(3-hydroxy-phenyl)-pyrimidine-5-carbonitrile-4-sec-Butyl-2-(2,3-difluoro-benzyl sulfanyl)-6-hydroxy pyrimidine-5-carbonitrile,
2-(2,3-Difluoro-benzylsulfanyl)-4-hydroxy-6-(1-phenyl-ethyl)-pyrimidine-5-carbonitrile,
4-Cyclopropylmethyl-2-(2,3-difluoro-benzylsulfanyl)-6 hydroxy-pyrimidine-5-carbonitrile,
Trans-2-[5-Cyano-2-(2,3-difluoro-benzylsulfanyl)-6 hydroxy-pyrimidin-4-yl]cyclopropane carboxylic acid,
(1R,2R)-2-[5-Cyano-2-(2,3-difluoro-benzylsulfanyl)-6 hydroxy-pyrimidin-4-yl]cyclopropane carboxylic acid,
(1R,2R)-2-[5-Cyano-2-(2,3-difluoro-benzylsulfanyl)-6 hydroxy-pyrimidin-4-yl]cyclopropane carboxylic acid,
Trans-2-(2,3-Difluoro-benzylsulfanyl)-4-hydroxy-6-(2-hydroxymethyl-cyclopropyl)-pyrimidine-5-carbonitrile,
Trans-2-[5-Cyano-2-(2,3-difluoro-benzyl sulfanyl)-6-hydroxy-pyrimidin-4-yl]cyclopropane carboxylic acid ethyl-(2-methyl-allyl)-amide,
2-[5-Cyano-2-(2,3-difluoro-benzylsulfanyl)-6-hydroxy-pyrimidin-4-yl]-cyclopropane carboxylic acid ethyl-methyl-amide,
2-[5-Cyano-2-(2,3-difluoro-benzylsulfanyl)-6-hydroxy-pyrimidin-4-yl]-cyclopropane carboxylic acid cyclopropyl methyl-amide,
2-(2,3-Difluoro-benzylsulfanyl)-4-[2-(2-ethyl-pyrrolidine-1carbonyl)-cyclopropyl]-6-hydroxypyrimidine-5-carbonitrile,
2-[5-Cyano-2-(2,3-difluoro-benzylsulfanyl)-6-hydroxy-pyrimidin-4-yl]-cyclopropane carboxylic acid (1-methoxymethyl-propyl)-amide,
2-[5-Cyano-2-(2,3-difluoro-benzylsulfanyl)-6-hydroxy-pyrimidin-4-yl]-cyclopropane carboxylic acid phenethyl-amide,
2-[5-Cyano-2-(2,3-difluoro-benzylsulfanyl)-6-hydroxy-pyrimidin-4-yl]-cyclopropane carboxylic acid 2-methoxy-benzylamide,
2-(2,3-Difluoro-benzylsulfanyl)-4-hydroxy-6-[2-(3-hydroxy-piperidine-1-carbonyl)cyclopropyl]-pyrimidine-5-carbonitrile,
2-[5-Cyano-2-(2,3-difluoro-benzylsulfanyl)-6-hydroxy-pyrimidin-4-yl]-cyclopropane carboxylic acid (pyridin-3-ylmethyl)-amide,
Trans-({2-[5-Cyano-2-(2,3-difluoro-benzylsulfanyl)-6-hydroxy-pyrimidin-4-yl]-cyclopropane carbonyl}-amino)-acetic acid ethyl ester,
2-(2,3-Difluoro-benzylsulfanyl)-4-hydroxy-6-[2-(morpholine-4-carbonyl)-cyclopropyl]pyrimidine-5-carbonitrile,
2-[5-Cyano-2-(2,3-difluoro-benzylsulfanyl)-6-hydroxy-pyrimidin-4-yl]-cyclopropane carboxylic acid (furan-2-ylmethyl)-amide,
2-[5-Cyano-2-(2,3-difluoro-benzylsulfanyl)-6-hydroxy-pyrimidin-4-yl]-cyclopropane carboxylic acid amide,
Trans-4-{2-[5-Cyano-2-(2,3-difluoro-benzylsulfanyl)-6-hydroxy-pyrimidin-4-yl]-cyclopropane carbonyl}-piperazine-1-carboxylic acid tert-butyl ester,
Trans-({2-[5-Cyano-2-(2,3-difluoro-benzylsulfanyl)-6-hydroxy-pyrimidin-4-yl]-cyclopropane carbonyl}-amino)-acetic acid,
2-(2,3-Difluoro-benzylsulfanyl)-4-hydroxy-6-[(R)-2-(5-methyl-furan-2-yl)-propyl]-pyrimidine-5-carbonitrile,
(1S,2S)-2-(2,3-Difluoro-benzylsulfanyl)-4-hydroxy-6-(2-hydroxymethyl-cyclopropyl)-pyrimidine-5-carbonitrile,
2-(2,3-Difluoro-benzylsulfanyl)-4-hydroxy-6-[(R)-1-(5-methyl-furan-2-yl)-propylamino]-pyrimidine-5-carbonitrile and
(1R,2R)-2-(2,3-Difluoro-benzylsulfanyl)-4-hydroxy-6-(2-hydroxymethyl-cyclopropyl)-pyrimidine-5-carbonitrile.

7. (canceled)

8. A pharmaceutical composition comprising a compound of claim 6 together with at least: one pharmaceutically acceptable excipient.

9. A method for the prevention or treatment of a CXCR2 receptor mediated condition or disease comprising administering an effective amount of at least one compound of claim 6 to a subject in need of such treatment.

10. A method of claim 9 wherein the condition or disease is an inflammatory or allergic condition, particularly an inflammatory or obstructive airway disease.

11. A method of claim 5 wherein the condition or disease is an inflammatory or allergic condition, particularly an inflammatory or obstructive airway disease.
